(19) United States
(12) Patent Application Publication
(10) Pub. No.: US 2012/0128118 A1
(43) Pub. Date: May 24, 2012

(54) X-RAY SYSTEM AND METHOD TO GENERATE X-RAY IMAGE DATA

(76) Inventors: Rainer Graumann, Hoechstadt (DE); Volker Heer, Gundelsheim (DE)

(21) Appl. No.: 13/302,237

(22) Filed: Nov. 22, 2011

(30) Foreign Application Priority Data
Nov. 24, 2010 (DE) 10 2010 061 882.9

(51) Int. Cl. A61B 6/03

(52) U.S. Cl. 378/9

(57) ABSTRACT

An x-ray system to generate x-ray image data of a predefined volume segment of an examination subject has either an arc-shaped mount or an annular gantry, an x-ray emitter arrangement with multiple x-ray microemitters, an x-ray detector arrangement with multiple x-ray pixels arranged directly adjacent to one another, and a controller to activate the x-ray emitter arrangement and the x-ray detector arrangement. The x-ray emitter arrangement and the x-ray detector are situated opposite one another on the arc-shaped mount or the gantry. The x-ray system is designed for introduction of the examination subject between the x-ray emitter arrangement and the x-ray detector.

### BACKGROUND OF THE INVENTION

1. Field of the Invention

The present invention concerns an x-ray system (for example a C-arm x-ray system or an O-arm x-ray system) that has an x-ray radiation source and an x-ray detector cooperating with this, and a corresponding method to generate x-ray image data.

2. Description of the Prior Art

According to the prior art, x-ray vacuum tubes are used as radiation sources in medical x-ray systems. Free electrons are released by the tube current that flows through the glow filament and they are accelerated by the application of the tube voltage between the cathode and the anode. Bremsstrahlung (which essentially corresponds to the x-ray radiation) arises as a result in the focus of the anode. Due to the focusing of the electron beam onto the anode, the x-ray focus has an extent of approximately one millimeter. Therefore it can be viewed as a point shape for most problems. The x-ray dose is determined by the level of the tube current and the tube voltage and by a pre-filtering that serves to filter out low-energy portions in the x-ray radiation that are not effective for imaging.

According to the prior art, it is a problem that the size and shape of the x-ray vacuum tube significantly determines the system geometry of the x-ray system.

### SUMMARY OF THE INVENTION

An object of the present invention is to provide x-ray systems that have the same or more functionality than corresponding known systems given smaller external dimensions.

Within the scope of the present invention, an x-ray system is provided to generate x-ray image data of a predefined volume segment of an examination subject. The x-ray system has an arc-shaped mount (also known as a C-arm); an x-ray emitter arrangement with multiple x-ray microemitters; an x-ray detector arrangement, such as a flat panel x-ray detector, that comprises multiple x-ray pixels arranged directly next to one another; and a controller in order to control the x-ray emitter arrangement and the flat panel x-ray detector. The x-ray emitter arrangement and the x-ray detector are situated opposite one another on the arc-shaped mount. Moreover, the x-ray system is designed for introduction of the examination subject between the x-ray emitter arrangement and the x-ray detector.

According to the invention, the arrangement of x-ray microemitters means x-ray microemitters that are produced in a semiconductor technique and over a large area and in matrix structure. The arrangement of x-ray microemitters, which is also designated as a flat panel x-ray emitter, enables a parallel beam geometry and an individual activation of the individual x-ray microemitters or emitter cells (for example, the x-ray radiation can be individually adjusted differently for each x-ray microemitter). The x-ray pixels arranged next to one another are cells within the scope of the invention that are designed in a semiconductor technique and are produced over a large area and in a matrix structure. Each x-ray pixel includes a photodiode which, depending on the radiated x-ray radiation, generates an electrical charge that is stored and read out. The arrangement of the x-ray pixels or the x-ray detector arrangement is also known as a flat panel x-ray detector.

In particular, due to the low structural depth of the x-ray emitter arrangement the arc-shaped mount (the C-arm, for example) can be significantly reduced in size in comparison to the prior art given a constant free opening for the patient to be examined.

The x-ray system according to the invention can be fashioned in a configuration known as a non-isocentric design, wherein the x-ray system is designed for an orbital rotation of the arc-shaped mount in a rotation angle of more than 180°.

While—given an isocentric design—the central beam of the x-ray emitter arrangement always travels through the rotation center of the arc-shaped mount, independently of the rotation angle which the arc-shaped mount has in the orbital rotation, this is not the case given a non-isocentric design. Given a non-isocentric design, the central beam wanders out of the rotation center at defined rotation angles.

In a preferred embodiment according to the invention, the total area of the x-ray detector (i.e. the total area of the x-ray pixels arranged like matrix) essentially provides a lateral surface of a maximum volume for the imaging of which the x-ray system is designed.

To acquire x-ray image data of a three-dimensional volume, multiple x-ray exposures of the volume are created from different directions (with different orbital rotation angles). The area of the x-ray detector corresponds approximately to an exposed area in the volume due to the parallel beam geometry of the x-ray emitter arrangement. For this reason, assuming that the area of the x-ray emitter arrangement is at least not smaller than the area of the x-ray detector, a volume whose lateral surface is equal to the area of the x-ray detector can be approximately acquired by means of the three-dimensional imaging. For example, if the x-ray detector has a quadratic area of edge length a, approximately a volume a³ can be acquired by means of the three-dimensional imaging.

The volume that can be reconstructed by means of the three-dimensional imaging of the x-ray system according to the invention is therefore larger by a factor of 8 than a volume of an x-ray system with identical x-ray detector surface according to the prior art. Due to the point-shaped beam geometry according to the prior art, given a volume arranged at the rotation center or isocenter of the arc-shaped mount, the area from which x-rays exit from the volume and strike the x-ray detector is smaller by a factor of 2 than the area of the x-ray detector. Since this factor of 2 is effective in all three spatial directions, the aforementioned factor of 8 results from 2³ for quadratic x-ray detectors, for example.

Expressed differently, with an x-ray system according to the invention a volume that is 8 times greater can be reconstructed given the same x-ray detector area, in spite of the smaller dimensions of the x-ray system.

According to a further embodiment of the invention, the x-ray system is designed to acquire geometry data of subjects within the predetermined volume segment. For this purpose the x-ray system acquires a two-dimensional x-ray image of the predetermined volume segment in which the corresponding subjects are irradiated and—based on this x-ray image—acquires and measures length dimensions and angle with regard to these subjects in the acquired x-ray image. The values measured in the x-ray image for a length or an angle can be converted into the values for the length and the angle with regard to the subject, independent of the distance between the flat panel x-ray detector and the subject and/or independent of the distance between the subject and the flat panel x-ray emitter. It must be insured that projections of subjects onto the x-ray detector are shown, and thus measured. A measured length or a measured angle corresponds precisely to the corresponding length or, respectively, the corresponding angle of the projection of the subject.

For example, if an angle is known with which the subject is tilted relative to the x-ray detector, the length of the subject can then be determined directly from the measured length of the projection of the subject under consideration of the known angle, without the clearance between subject and x-ray detector still having to be determined, for example. This is disadvantageously not possible with a point-beam geometry as it applies in conventional x-ray systems.

Due to the parallel beam geometry (mentioned in the preceding) of the x-ray emitter arrangement used according to the invention, length dimensions and angles with regard to the subjects in the volume segment can be determined from the length dimensions of a projection of subjects and from the angles with regard to these subjects, without the distance between subject and x-ray detector needing to be known for this. For example, given knowledge of the attitude of the subjects, the length dimensions and angles of the subjects are determined from the lengths and angles measured in the x-ray image. In other words, the present invention utilizes the parallel beam geometry in order to acquire the corresponding geometry data with regard to the subjects. Such a geometry data acquisition in conventional x-ray systems is not possible due to the point-shaped radiation source and due to ignorance of how far irradiated subjects are from the x-ray detector.

A further significant advantage relative to point beam geometry is that subjects are always shown the same independent of their attitude and do not change their shape, for example.

According to a further embodiment according to the invention, the x-ray system is designed to generate a three-dimensional image data set of the predetermined volume segment. The x-ray detector is attached in a fixed—i.e. stationary—manner to the arc-shaped mount (to one end of the C-arm, for example). The x-ray microemitters in this embodiment form a surface which is larger than the area of the x-ray detector. The x-ray system generates two-dimensional x-ray images of the predetermined volume segment in that, in each acquisition of such a two-dimensional x-ray image, the x-ray system activates a different set of these x-ray microemitters or partial area of the x-ray emitter arrangement to generate x-rays which then respectively expose the predetermined volume segment from a different angle. The x-ray system generates the three-dimensional image data set from the two-dimensional x-ray images.

Because the x-ray microemitters are fashioned on an area which is larger than the area of the x-ray detector, opposite the x-ray detector on the arc-shaped mount, the predetermined volume segment can advantageously be exposed from different angles. The different two-dimensional x-ray image exposures for generation of a three-dimensional image data set can therefore be generated more quickly than is the case given an x-ray system according to the prior art, in which an orbital rotation of the C-arm has to take place in order to generate an x-ray image from a different angle. For example, tomosynthesis exposures can be generated via this embodiment without generating a rotation of the arc-shaped mount.

Within the scope of the present invention, an additional x-ray system is also provided for generation of x-ray image data of a predetermined volume segment of an examination subject. This x-ray system has an annular gantry, an x-ray emitter arrangement with multiple x-ray microemitters, an x-ray detector arrangement or, respectively, an x-ray detector with multiple x-ray pixels arranged directly adjacent to one another, and a controller to activate the x-ray emitter arrangement and the x-ray detector arrangement. The x-ray emitter arrangement is arranged opposite the x-ray detector arrangement on the gantry. The gantry is designed for introduction of the examination subject.

The gantry (also known as an O-arm) defines a type of annular tunnel (or torus) in which both the x-ray emitter arrangement and the x-ray detector orbit the examination subject (and therefore the volume segment to be acquired) in order to create x-ray image exposures of the predetermined volume segment from different orbital rotation angles. In the further x-ray system the gantry plays a role similar to the arc-shaped mount (the C-arm) in the x-ray system described in the preceding.

Due to the flat x-ray emitter arrangement according to the invention, a closed O-arm (gantry) can be designed a great deal smaller, more compact and more flexible in comparison to x-ray systems according to the prior art.

For example, the area of the x-ray emitter arrangement can be arranged distributed over the entire gantry (the entire O-arm), such that only the x-ray detector is movable along the gantry. For this, to create two-dimensional x-ray images of the predetermined volume segment from different orbital rotation angles only the x-ray detector is moved accordingly within the gantry, and corresponding x-ray microemitters of the x-ray emitter arrangement (that is situated opposite the x-ray detector) are activated to acquire the respective x-ray image.

While, according to the prior art, both the x-ray source and the x-ray detector are thus rotated around predetermined volume segment within the gantry, according to this embodiment only the x-ray detectors must be moved. The activation of the corresponding emitter components—i.e. the corresponding x-ray microemitters—then takes place corresponding to the respective x-ray detector position. The area of the x-ray microemitter respectively activated per x-ray image in particular respectively corresponds to the area of the x-ray detector.

Moreover, the x-ray detector can also be arranged distributed over the entire gantry (the entire O-arm). For example, the x-ray microemitters and the x-ray pixels can be arranged annularly (for example in the form of two rings situated in parallel with the same axis of symmetry) in the gantry. To create a two-dimensional x-ray image of the predetermined volume segment from a defined orbital rotation angle, for this x-ray microemitters corresponding to the orbital rotation angle are activated and the measurement signals of x-ray pixels opposite these x-ray microemitters in the gantry (for example offset by 180° opposite said x-ray microemitters) are detected in order to construct the x-ray image from these. For this purpose, the x-ray microemitters or collimators are aligned at a slight angle so that their generated radiation strikes the (correspondingly slightly angled) opposite x-ray pixels.

In this embodiment neither the x-ray source nor the x-ray detector must thus be rotated to acquire multiple x-ray images from different viewing angles.

Furthermore, multiple such parallel ring arrangements that each include annularly arranged x-ray microemitters and annularly arranged x-ray pixels parallel thereto can be arranged adjacent to one another, with the same axis of symmetry. Multiple such parallel ring arrangements enable a volume scanning of the predetermined volume segment.

According to one embodiment of the further x-ray system, the total area of the x-ray detector essentially corresponds to a lateral surface of a maximum volume for whose three-dimensional imaging the further x-ray system is designed.

Moreover, the further x-ray system can be designed for geometry data acquisition of subjects within the predetermined volume segment.

The two embodiments of the further x-ray system that are described in the preceding can each be modified according to the embodiments of the x-ray system that have been explained in detail above.

Within the scope of the present invention, a different x-ray system is provided to generate x-ray image data of a predetermined volume segment of an examination subject. This different x-ray system comprises an x-ray emitter arrangement with multiple x-ray microemitters, an x-ray detector arrangement or, respectively, an x-ray detector with multiple x-ray pixels arranged immediately adjacent to one another, and a controller to activate the x-ray emitter arrangement and the x-ray detector arrangement. In the different x-ray system, the x-ray emitter arrangement and the x-ray detector arrangement are arranged stationary, situated opposite one another.

This different x-ray system according to the invention can also be designed with significantly smaller external dimensions in comparison to a known x-ray system with the same clearance between x-ray source and detector, in particular due to the smaller structural depth of the x-ray emitter arrangement.

The different x-ray system can also have the two embodiments described in the preceding for generation of the three-dimensional image data set and for geometry data acquisition, such that the corresponding embodiments of the x-ray system are referenced for a more precise description of these embodiments.

According to one embodiment of the different x-ray system according to the invention, the different x-ray system is designed to generate a three-dimensional image data set. The x-ray microemitters are arranged distributed on an area which is larger than the total area of the x-ray detector. Two-dimensional x-ray images of the predetermined volume segment are created by the different x-ray system in that a different set (a different area) of x-ray microemitters is respectively activated in order to generate respective x-rays which expose the predetermined volume segment from a different angle. The three-dimensional image data set is generated from the two-dimensional x-ray images generated in such a manner.

Because the x-ray emitter arrangement which is situated opposite the x-ray detector has a total area which is in particular larger than the area of the x-ray detector, x-ray images from different viewing angles can be generated without a movement of the x-ray emitter arrangement or the x-ray detector. For this purpose, x-ray microemitters at a point associated with the corresponding viewing angle are activated which in particular take up an area that corresponds to the area of the x-ray detector. This embodiment according to the invention accordingly enables the acquisition and reconstruction of tomosynthesis images without moving components, for example.

Within the scope of the present invention, a method is also provided to generate x-ray image data of a predetermined volume segment of an examination subject by means of an x-ray system that includes an arc-shaped mount, an x-ray emitter arrangement with multiple x-ray emitters, an x-ray detector arrangement with multiple x-ray pixels arranged directly adjacent to one another, and a controller to activate the x-ray emitter arrangement and the x-ray detector arrangement. The method includes the following steps:
Arrange the x-ray emitter arrangement and the x-ray detector arrangement at opposite points on the arc-shaped mount.
Activate corresponding x-ray microemitters in order to expose the volume segment which is situated between the x-ray emitter arrangement and the x- ray detector arrangement by means of x-rays generated in such a manner.
Detect the x-rays which have exposed the predetermined volume segment by means of the x-ray detector arrangement in order to generate the x-ray image data.

Within the scope of the present invention, an additional method is provided to generate x-ray image data of a predetermined volume segment of an examination subject by means of an additional x-ray system. The additional x-ray system includes an annular gantry (an O-arm, for example), an x-ray emitter arrangement with multiple x-ray microemitters, an x-ray detector arrangement with multiple x-ray pixels arranged immediately adjacent to one another, and a controller to activate the x-ray emitter arrangement and the x-ray detector arrangement. This method includes the following steps:
The x-ray emitter arrangement and the x-ray detector arrangement are arranged at opposite points within the gantry.
Corresponding x-ray microemitters of the x-ray emitter arrangement are activated in order to expose the volume segment (which is situated between the x-ray emitter arrangement and the x-ray detector arrangement) by means of x-rays.
The x-rays emanating from the volume segment, which x-rays have previously exposed the volume segment, are detected by means of the x-ray detector arrangement in order to thereby generate the x-ray image data.

Within the scope of the present invention yet another method is provided to generate x-ray images of a predetermined volume segment of an examination subject by means of a different x-ray system. This different x-ray system has an x-ray emitter arrangement with multiple x-ray microemitters, an x-ray detector arrangement with multiple x-ray pixels arranged immediately adjacent to one another, and a controller to activate the x-ray emitter arrangement and the x-ray detector arrangement. This method includes the following steps:
Activate the x-ray emitter arrangement in order to expose the predetermined volume segment which is arranged between the x-ray emitter arrangement and the x-ray detector arrangement by means of x-rays.
Detect the x-rays which have exposed the predetermined volume segment by means of the x-ray detector arrangement in order to generate the x-ray image data.

The advantages of the method according to the invention that are described in the preceding essentially correspond to the advantages of the corresponding x-ray systems according to the invention.

Furthermore, the present invention encompasses a non-transitory, computer-readable data storage medium that is encoded with programming instructions such as a computer program or a software, which can be loaded into a memory of a programmable controller or a computer of an x-ray system. All or various described embodiments of the method according to the invention can be executed when the computer program runs in the controller or control device of the x-ray system. The programming instructions may require program means (libraries and auxiliary functions, for example) in order to realize the corresponding embodiments of the method. The programming instructions can be in source code (C++, for example) that must still be compiled (translated) and linked or that only must be interpreted, or an be an executable software code that need only be loaded into the corresponding computer for execution.

The data storage medium can be a DVD, a magnetic tape or a USB stick—on which is stored electronically readable control information.

The present invention is suitable for x-ray systems used in the medical field, but the present invention is not limited to this preferred field of application since, due to their advantageously small dimensions, x-ray systems according to the invention are generally also usable in any other field in which subjects are exposed with x-rays for analysis (for example in crystal structure analysis).

### BRIEF DESCRIPTION OF THE DRAWINGS

FIG. 1 shows a C-arm x-ray system according to the invention.

FIG. 2 shows an x-ray emitter arrangement according to the invention.

FIG. 3 shows an additional embodiment of a C-arm x-ray system according to the invention.

FIG. 4 shows an O-arm x-ray system according to the invention.

FIG. 5 shows an additional embodiment of an O-arm x-ray system according to the invention.

FIG. 6 shows a stationary x-ray system according to the invention.

### DESCRIPTION OF THE PREFERRED EMBODIMENTS

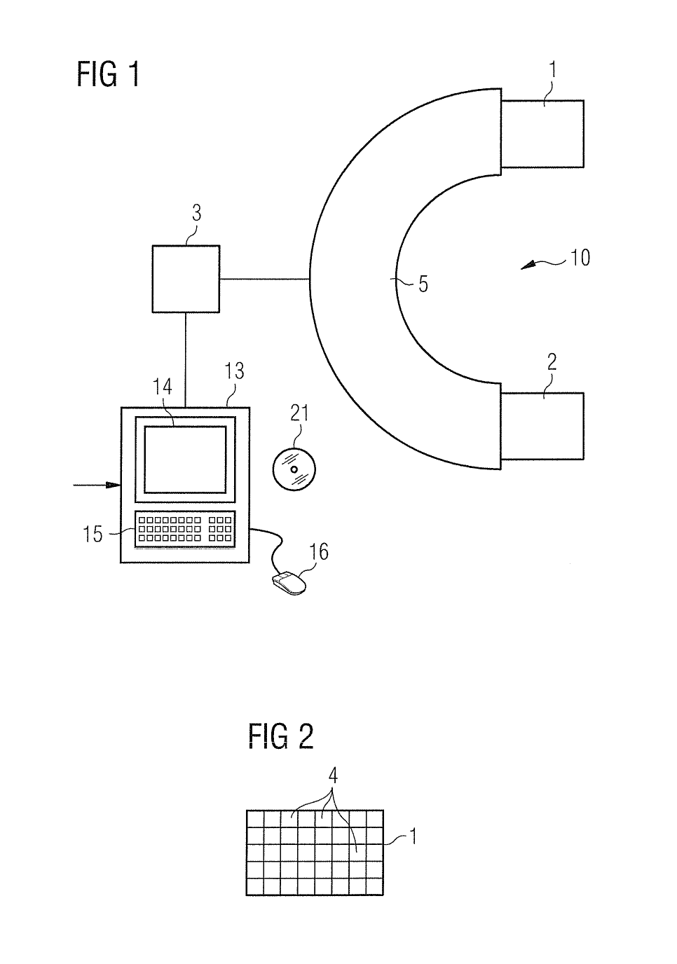

Shown in FIG. 1 is a first embodiment of a C-arm x-ray system 10 according to the invention. The C-arm x-ray system 10 has a flat panel x-ray emitter 1 and a flat panel x-ray detector 2 which are mounted on a C-arm 5. Moreover, the C-arm x-ray system 10 has a controller 3 to control the flat panel x-ray emitter 1 and the flat panel x-ray detector 2, and to rotate the C-arm 5 and a terminal 13 with monitor 14, keyboard 15, mouse 16, and a DVD 21.

In order to generate x-ray image data of a volume segment of an examination subject, the examination subject is arranged within the C-arm 5 such that the volume segment is situated between flat panel x-ray emitter 1 and flat panel x-ray detector 2. The x-rays (which are radiated traveling in parallel from the x-ray microemitters of the flat panel x-ray emitter 1) expose the predetermined volume segment and are then detected by the x-ray pixels of the flat panel x-ray detector 2. The x-ray image data of the predetermined volume segment are then reconstructed from the data of the flat panel x-ray detector 2.

In order to generate x-ray images from different viewing angles relative to the volume segment, the C-arm 5 is rotated orbitally, meaning that the rotation axis is situated perpendicular to the plane of the drawing. Since the flat panel x-ray emitter 1 and the flat panel x-ray detector 2 are firmly attached to the C-arm 5, the flat panel x-ray emitter 1 and the flat panel x-ray detector 2 are rotated by the same rotation angle so that x-rays generated by the flat panel x-ray emitter 1 in turn strike orthogonally on the flat panel x-ray detector 2, independent of the rotation angle.

The x-ray image data are prepared by the controller 3 and shown on the monitor 14 depending on specific instructions which are input via the keyboard 15 and the mouse 16.

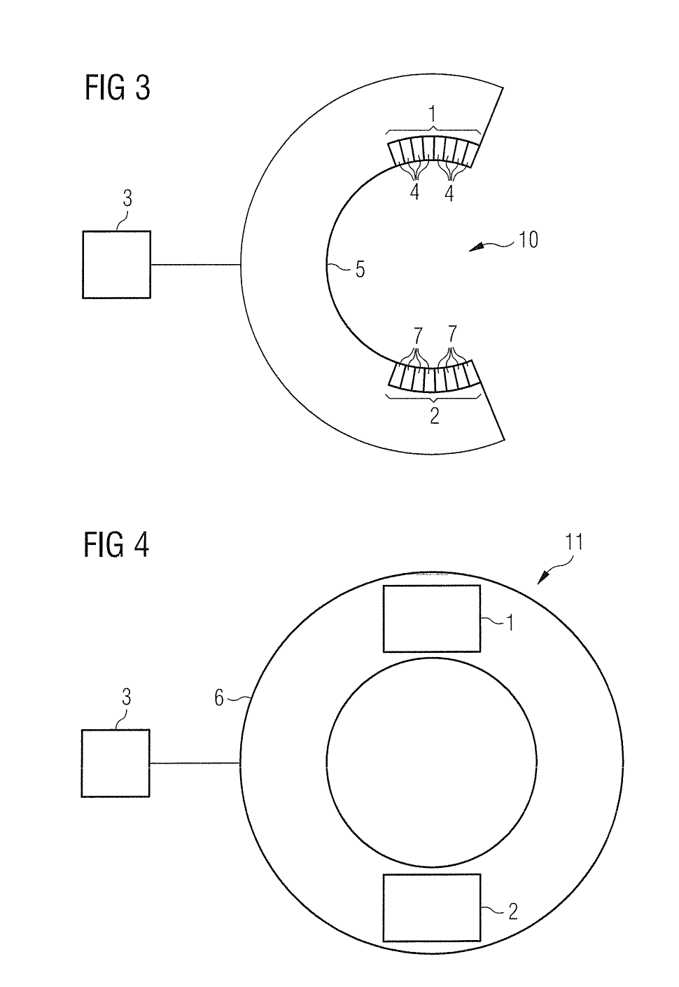

Schematically shown in FIG. 2 is a flat panel x-ray emitter 1 which comprises an arrangement of multiple (40 in FIG. 2) x-ray microemitters 4. Each x-ray microemitter 4 has dimensions of approximately 1-10 mm² which in particular correspond to dimensions of an x-ray pixel 7 (see FIG. 3). The flat panel x-ray emitter 1 generates a laminar x-ray radiation (the x-rays of the individual x-ray microemitters travel parallel to one another), which is contrasted with the conical radiation of an x-ray vacuum tube used presently. For clarification it is noted that a classical vacuum tube radiates isotropically in a point shape and within wide boundaries. The conical radiation arises in that only a small x-ray window is opened. The remainder is shielded. The x-ray window thereby depends on the size and distance of the detector.

An additional embodiment of a C-arm x-ray system 10 according to the invention is shown in FIG. 3. In this embodiment the x-ray microemitters 4 of the flat panel x-ray emitter 1 are arranged across the extent of the C-arm 5 on an area which is larger than the area of the flat panel x-ray detector 2 with this x-ray system 10, the predetermined volume segment can be exposed from various angles even without a rotation of the C-arm 5 in that a different partial area of the flat panel x-ray emitter 1 is respectively activated. The flat panel x-ray emitter 1 (i.e. the arrangement of the x-ray microemitters) is thereby advantageously arranged symmetrical (in particular axially symmetrical relative to the rotation axis of the C-arm 5) to the flat panel x-ray detector 2 (i.e. to the arrangement of the x-ray pixels 7).

Moreover, an orbital rotation of the C-arm 5 can be implemented in order to correspondingly increase the angle from which the predetermined volume segment is exposed.

Shown in FIG. 4 is a first embodiment of an O-arm x-ray system 11 according to the invention. Instead of a C-arm 5, the O-arm x-ray system 11 has a gantry 6 in the form of a torus. In this gantry 6 the flat panel x-ray emitter 1 and the flat panel x-ray detector 2 are arranged opposite one another (thus offset from one another by 180°) such that they can rotate, wherein the arrangement comprising the flat panel x-ray emitter 1 and the flat panel x-ray detector 2 can be rotated arbitrarily in the gantry. The gantry 6 can be opened on one side in order to shift the gantry 6 across the patient to be examined or, respectively, the table on which the patient lies. The gantry 6 is closed again after the patient is located within said gantry 6.

In comparison to a C-arm x-ray system 10, the O-arm x-ray system 11 has the following advantages:
A rotation of the flat panel x-ray emitter 1 and the flat panel x-ray detector 2 by 360° is possible.
It is a very stable system.
No moving parts exist outside of the gantry 6, such that fewer problems with sterility occur.

With regard to the O-arm x-ray system 11, the size and weight on the one hand and the lesser flexibility given use as a radioscopy system on the other hand are to be cited as disadvantages relative to a C-arm x-ray system 10.

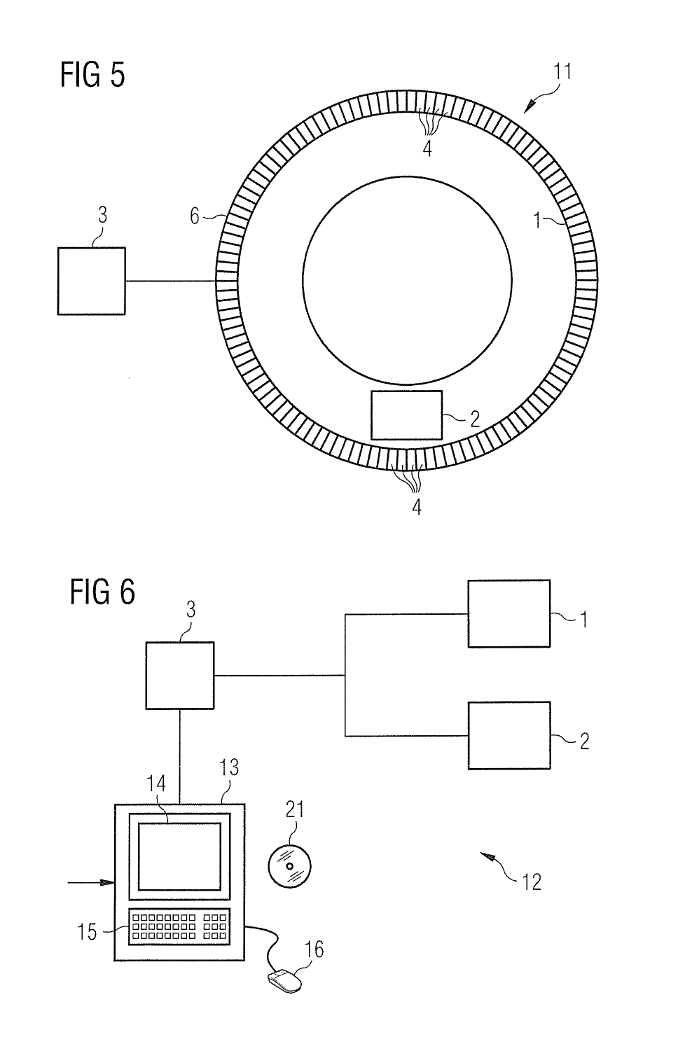

A second embodiment of an O-arm x-ray system 11 according to the invention is shown in FIG. 5. In this embodiment, the x-ray microemitters 4 are distributed across the entire area of the gantry 6. Therefore, to create an x-ray image only the flat panel x-ray detector 2 advantageously needs to be moved corresponding to the angle at which the x-ray image is to be created.

In the embodiment shown in FIG. 5, it is also possible that the flat panel x-ray detector 2 is not moved for the generation of multiple x-ray images with different viewing angles. To create a respective one of these x-ray images, x-ray microemitters 4 which are offset by 180°±“defined angle” (45°, for example) relative to the position of the flat panel x-ray detector 2 are thereby activated and form an area which essentially corresponds to the area of the flat panel x-ray detector 2.

The embodiment of a stationary x-ray system 12 according to the invention is shown in FIG. 6. In a stationary x-ray system 12, the flat panel x-ray emitter 1 and the flat panel x-ray detector 2 are arranged stationary (i.e. immobile).

In order to create x-ray images from different viewing angles even given a stationary x-ray system 12, the area of the flat panel x-ray emitter 1 is larger than the area of the flat panel x-ray detector 2. If the individual x-ray microemitters of the flat panel x-ray emitter 1 are respectively aligned relative to the flat panel x-ray detector 2, a partial area of the flat panel x-ray emitter 1 can be activated to create an x-ray image from a respective viewing angle. Even given a stationary x-ray system 12, it is thereby possible to acquire and reconstruct tomosynthesis images (for example) without moving components.

Although modifications and changes may be suggested by those skilled in the art, it is the intention of the inventors to embody within the patent warranted hereon all changes and modifications as reasonably and properly come within the scope of their contribution to the art.

We claim as our invention:

1. An x-ray system comprising:
an arc-shaped mount;
an x-ray emitter arrangement and an x-ray detector situated opposite each other on said arc-shaped mount with a spacing therebetween allowing introduction of an examination subject between said x-ray emitter arrangement and said x-ray detector;
said x-ray emitter arrangement comprising multiple x-ray microemitters;
said x-ray detector arrangement comprising multiple x-ray pixels arranged directly adjacent to one another; and
a control unit configured to activate the x-ray emitter arrangement to emit x- rays that irradiate a predetermined volume segment of the examination subject, said x-rays attenuated by said predetermined volume segment being detected by said x-ray detector arrangement.

2. An x-ray system as claimed in claim 1 wherein said arc-shaped mount is configured to execute an orbital rotation around a rotation center through an angle of more than 180°, and wherein said arc-shaped mount and said x-ray emitter arrangement are configured with a non-isocentric design in which a central x-ray beam emitted by said x-ray emitter arrangement wanders out of said rotation center during said orbital rotation of said arc-shaped mount.

3. An x-ray system as claimed in claim 1 wherein said x-ray detector arrangement has a total area in which said x-rays are detected configured to provide a lateral surface having a maximum volume for three-dimensional imaging of said examination subject.

4. An x-ray system as claimed in claim 1 wherein said control unit is configured to operate said x-ray emitter arrangement and said x-ray detector arrangement to acquire geometry data of said predetermined volume segment of the examination subject by generating a two-dimensional x-ray image of said predetermined volume segment, and comprising a processor configured to detect and measure lengths and angles of objects within said two-dimensional x-ray image.

5. An x-ray system as claimed in claim 1 wherein:
said x-ray detector arrangement is rigidly attached to said arc-shaped mount, and has a total detector area;
said x-ray microemitters of said x-ray emitter arrangement are distributed on a surface of said x-ray emitter arrangement that is larger than said total detector area of said x-ray detector arrangement;
said control unit is configured to operate said x-ray emitter arrangement, said x-ray detector arrangement and said arc-shaped mount to generate a plurality of two-dimensional x-ray images of said predetermined volume segment by activating said x-ray microemitters of said x-ray emitter arrangement to generate x-rays that respectively irradiate the predetermined volume segment from different angles for the respective two-dimensional x-ray images; and
a processor supplied with said two-dimensional x-ray images that is configured to generate a three-dimensional image data set of said predetermined volume segment from said plurality of two-dimensional x-ray images.

6. An x-ray system comprising:
an annular gantry;
an x-ray emitter arrangement and an x-ray detector arrangement situated opposite each other on said gantry with a spacing therebetween, said spacing and said gantry being configured to allow introduction of an examination subject in said gantry between said x-ray emitter arrangement and said x-ray detector arrangement;
said x-ray emitter arrangement comprising multiple x-ray microemitters;
said x-ray detector arrangement comprising multiple x-ray pixels arranged directly adjacent to one another; and
a control unit configured to activate the x-ray emitter arrangement and the x-ray detector arrangement to irradiate a predetermined volume segment of the examination subject with x-rays emitted by said x-ray emitter arrangement, said x-rays attenuated by the examination subject being detected by the x-ray detector arrangement.

7. An x-ray system as claimed in claim 6 wherein:
said x-ray microemitters are mounted immobily on said gantry and are distributed around an entirety of said gantry;
said x-ray detector arrangement is movable along said gantry; and
said control unit is configured to activate said x-ray microemitters to generate a plurality of two-dimensional x-ray images of the predetermined volume segment from different irradiation angles and to move the x-ray detector arrangement along the gantry to detect x-rays emitted by currently-activated x-ray microemitters.

8. An x-ray arrangement as claimed in claim 7 wherein:
said x-ray detector arrangement is mounted immobily on said gantry and is distributed around an entirety of said gantry; and
said control unit is configured to activate said x-ray microemitters to irradiate said predetermined volume segment from respective irradiation angles around said gantry, with said x-rays attenuated by the examination subject being detected by respective x-ray pixels situated opposite currently-activated x-ray microemitters.

9. An x-ray system as claimed in claim 6 wherein said x-ray detector arrangement has a total detector surface area representing a lateral surface of a maximum volume for obtaining a three-dimensional image of said predetermined volume segment.

10. An x-ray system as claimed in claim 6 wherein said control unit is configured to operate said x-ray emitter arrangement and said x-ray detector arrangement to generate a two-dimensional x-ray image of said predetermined volume segment, and wherein said x-ray system comprises a processor supplied with said two-dimensional x-ray image, said processor being configured to detect and measure lengths and angles of respective objects in said two-dimensional x-ray image.

11. An x-ray system comprising:
an x-ray emitter arrangement comprising multiple x-ray microemitters;
an x-ray detector arrangement comprising multiple x-ray pixels arranged adjacent to one another;
a mount on which said x-ray emitter arrangement and said x-ray detector arrangement are situated fixedly opposite to each other with a spacing therebetween allowing introduction of an examination subject between said x-ray emitter arrangement and said x-ray detector arrangement; and
a control unit configured to activate the x-ray emitter arrangement and the x-ray detector arrangement to irradiate a predetermined volume segment of the examination subject with x-rays emitted by at least some of said multiple x-ray microemitters, said x-rays attenuated by the examination subject being detector by said x-ray detector arrangement.

12. An x-ray system as claimed in claim 11 wherein said x-ray detector arrangement has a lateral detector surface maximized for three-dimensional imaging.

13. An x-ray system as claimed in claim 11 wherein said control unit is configured to operate said x-ray emitter arrangement and said x-ray detector arrangement to obtain a two-dimensional x-ray image of the predetermined volume segment, and wherein said x-ray system comprises a processor supplied with said two-dimensional x-ray image, said processor being configured to detect and measure lengths and angles of respective objects in said two-dimensional x-ray image.

14. An x-ray system as claimed in claim 11 wherein:
said x-ray detector arrangement has a total detector area;
said x-ray microemitters of said x-ray emitter arrangement are distributed on said mount in an area that is larger than said total detector area of said x-ray detector arrangement;
said control unit is configured to operate said x-ray emitter arrangement and said x-ray detector arrangement to generate a plurality of two-dimensional x-ray images of the predetermined volume segment by irradiating said predetermined volume segment from different angles by activating respectively different sets of said x-ray microemitters at the respectively different angles; and
said x-ray system comprises a processor supplied with said plurality of two-dimensional x-ray images, said processor being configured to generate a three-dimensional image data set from said plurality of two-dimensional x-ray images.

15. A method for operating an x-ray system comprising:
providing an x-ray emitter arrangement and an x-ray detector situated opposite each other on an arc-shaped mount with a spacing therebetween allowing introduction of an examination subject between said x-ray emitter arrangement and said x-ray detector;
forming said x-ray emitter arrangement of multiple x-ray microemitters;
forming said x-ray detector arrangement of multiple x-ray pixels arranged directly adjacent to one another; and
activating the x-ray emitter arrangement to emit x-rays that irradiate a predetermined volume segment of the examination subject, and detecting x-rays attenuated by said predetermined volume segment with said x-ray detector arrangement.

16. A method as claimed in claim 15 comprising moving said arc-shaped mount in an orbital rotation around a rotation center through an angle of more than 180°, and wherein said arc-shaped mount and said x-ray emitter arrangement are configured with a non-isocentric design thereby causing a central x-ray beam emitted by said x-ray emitter arrangement to wander out of said rotation center during said orbital rotation of said arc-shaped mount.

17. A method as claimed in claim 15 comprising providing said x-ray detector arrangement with a total area in which said x-rays are detected configured to provide a lateral surface having a maximum volume for three-dimensional imaging of said examination subject.

18. A method as claimed in claim 15 comprising operating said x-ray emitter arrangement and said x-ray detector arrangement to acquire geometry data of said predetermined volume segment of the examination subject by generating a two-dimensional x-ray image of said predetermined volume segment and, in a processor, detecting and measuring lengths and angles of objects within said two-dimensional x-ray image.

19. A method as claimed in claim 15 comprising:
rigidly attaching said x-ray detector arrangement to said arc-shaped mount, said x-ray detector arrangement having a total detector area;
distributing said x-ray microemitters of said x-ray emitter arrangement on a surface of said x-ray emitter arrangement that is larger than said total detector area of said x-ray detector arrangement;
operating said x-ray emitter arrangement, said x-ray detector arrangement and said arc-shaped mount to generate a plurality of two-dimensional x-ray images of said predetermined volume segment by activating said x-ray microemitters of said x-ray emitter arrangement to generate x-rays that respectively irradiate the predetermined volume segment from different angles for producing the respective two-dimensional x-ray images; and
in a processor supplied with said two-dimensional x-ray images, generating a three-dimensional image data set of said predetermined volume segment from said plurality of two-dimensional x-ray images.

20. A method for operating an x-ray system comprising:
providing an x-ray emitter arrangement and an x-ray detector arrangement situated opposite each other on an annular gantry with a spacing therebetween, said spacing and said gantry being configured to allow introduction of an examination subject in said gantry between said x-ray emitter arrangement and said x-ray detector arrangement;
forming said x-ray emitter arrangement of multiple x-ray microemitters;
forming said x-ray detector arrangement of multiple x-ray pixels arranged directly adjacent to one another; and
activating the x-ray emitter arrangement and the x-ray detector arrangement to irradiate a predetermined volume segment of the examination subject with x-rays emitted by said x-ray emitter arrangement, and detecting x-rays attenuated by the examination subject with the x-ray detector arrangement.

21. A method as claimed in claim 20 comprising:
mounting said x-ray microemitters immobily on said gantry and distributing said x-ray microemitters around an entirety of said gantry;
mounting said x-ray detector arrangement so as to be movable along said gantry; and
activating said x-ray microemitters to generate a plurality of two-dimensional x-ray images of the predetermined volume segment from different irradiation angles while moving the x-ray detector arrangement along the gantry to detect x-rays emitted by currently-activated x-ray microemitters.

22. A method as claimed in claim 20 comprising:
mounting said x-ray detector arrangement immobily on said gantry and distributing said x-ray detector arrangement around an entirety of said gantry; and
activating said x-ray microemitters to irradiate said predetermined volume segment from respective irradiation angles around said gantry, and detecting said x-rays attenuated by the examination subject with respective x-ray pixels situated opposite currently-activated x-ray microemitters.

23. A method as claimed in claim 20 comprising providing said x-ray detector arrangement with a total detector surface area representing a lateral surface of a maximum volume for obtaining a three-dimensional image of said predetermined volume segment.

24. A method as claimed in claim 20 comprising operating said x-ray emitter arrangement and said x-ray detector arrangement to generate a two-dimensional x-ray image of said predetermined volume segment and, in a processor supplied with said two-dimensional x-ray image, detecting and measuring lengths and angles of respective objects in said two-dimensional x-ray image.

25. A method for operating an x-ray system, comprising:
providing an x-ray emitter arrangement comprising multiple x-ray microemitters;
providing an x-ray detector arrangement comprising multiple x-ray pixels arranged adjacent to one another;
situating said x-ray emitter arrangement and said x-ray detector fixedly opposite to each other with a spacing therebetween allowing introduction of an examination subject between said x-ray emitter arrangement and said x-ray detector arrangement; and
activating said x-ray emitter arrangement and the x-ray detector arrangement to irradiate a predetermined volume segment of the examination subject with x-rays emitted by at least some of said multiple x-ray microemitters, and detecting x-rays attenuated by the examination subject with said x-ray detector arrangement.

26. An x-ray system as claimed in claim 25 comprising providing said x-ray detector arrangement with a lateral detector surface maximized for three-dimensional imaging.

27. An x-ray system as claimed in claim 25 comprising operating said x-ray emitter arrangement and said x-ray detector arrangement to obtain a two-dimensional x-ray image of the predetermined volume segment and in a processor supplied with said two-dimensional x-ray image, detecting and measuring lengths and angles of respective objects in said two-dimensional x-ray image.

28. An x-ray system as claimed in claim 25 comprising:
providing said x-ray detector arrangement with a total detector area;
distributing said x-ray microemitters of said x-ray emitter arrangement on said mount in an area that is larger than said total detector area of said x-ray detector arrangement;
operating said x-ray emitter arrangement and said x-ray detector arrangement to generate a plurality of two-dimensional x-ray images of the predetermined volume segment by irradiating said predetermined volume segment from different angles by activating respectively different sets of said x-ray microemitters at the respectively different angles; and
in a processor supplied with said plurality of two-dimensional x-ray images, generating a three-dimensional image data set from said plurality of two-dimensional x-ray images.

29. A non-transitory, computer-readable data storage medium encoded with programming instructions, said data storage medium being loaded into a computerized control and evaluation system of an x-ray system comprising an arc-shaped mount on which an x-ray emitter arrangement and an x-ray detector arrangement are situated opposite each other, said x-ray emitter arrangement comprising multiple x-ray microemitters and said x-ray detector arrangement comprising multiple x-ray pixels arranged immediately adjacent one another, said programming instructions causing said computerized control and evaluation system to:
activate the x-ray emitter arrangement to emit x-rays that irradiate a predetermined volume segment of the examination subject, said x-rays attenuated by said predetermined volume segment being detected by said x-ray detector arrangement.

30. A non-transitory, computer-readable data storage medium encoded with programming instructions, said data storage medium being loaded into a computerized control and evaluation system of an x-ray system comprising a gantry on which an x-ray emitter arrangement and an x-ray detector arrangement are situated opposite each other, said x-ray emitter arrangement comprising multiple x-ray microemitters and said x-ray detector arrangement comprising multiple x-ray pixels arranged immediately adjacent one another, said programming instructions causing said computerized control and evaluation system to:
activate the x-ray emitter arrangement and the x-ray detector arrangement to irradiate a predetermined volume segment of the examination subject with x-rays emitted by said x-ray emitter arrangement, said x-rays attenuated by the -examination subject being detected by the x-ray detector arrangement.

31. A non-transitory, computer-readable data storage medium encoded with programming instructions, said data storage medium being loaded into a computerized control and evaluation system of an x-ray system comprising an mount on which an x-ray emitter arrangement and an x-ray detector arrangement are situated opposite each other, said x-ray emitter arrangement comprising multiple x-ray microemitters and said x-ray detector arrangement comprising multiple x-ray pixels arranged immediately adjacent one another, said programming instructions causing said computerized control and evaluation system to:
activate the x-ray emitter arrangement and the x-ray detector arrangement to irradiate a predetermined volume segment of the examination subject with x-rays emitted by at least some of said multiple x-ray microemitters, said x-rays attenuated by the examination subject being detector by said x-ray detector arrangement.
